(19) United States
(12) Patent Application Publication
(10) Pub. No.: US 2026/0110892 A1
(43) Pub. Date: Apr. 23, 2026

(54) MEDICAL MICROSCOPE AND MICROSCOPE SYSTEM

(71) Applicant: Jadent GmbH, Aalen (DE)

(72) Inventors: Klaus-Juergen JANIK, Neuler (DE); Ulrich WOESSNER, Geislingen (DE)

(73) Assignee: Jadent GmbH, Aalen (DE)

(21) Appl. No.: 19/327,652

(22) Filed: Sep. 12, 2025

Related U.S. Application Data
(63) Continuation of application No. PCT/EP2024/056816, filed on Mar. 14, 2024.

(30) Foreign Application Priority Data
Mar. 15, 2023 (DE) 10 2023 106 504.1

(51) Int. Cl. G02B 21/24; A61B 90/25; G02B 21/00; G02B 21/20

(52) U.S. Cl. CPC G02B 21/24; A61B 90/25; G02B 21/0012; G02B 21/20

(57) ABSTRACT

A medical microscope includes an eyepiece system having a binocular, an eyepiece base body supporting the binocular, and a suspension attached to the eyepiece base body securing the microscope to a support system. A microscope body has an objective lens for capturing light from a field of view associated with a microscope axis corresponding to a section of an optical symmetry axis extending from the microscope body to the field of view. An opto-mechanical system positioned between the eyepiece system and the microscope body channels light captured to the binocular. The opto-mechanical system allows pivoting movement of the microscope body about a pivot axis and has a rotation unit for moving the microscope body about a first axis of rotation extending coaxially, parallel or at an angle in the range of 0° to 5° to a section of the optical symmetry axis extending through the rotation unit.

### CROSS-REFERENCE TO RELATED APPLICATIONS

This application is a continuation under 35 U.S.C. § 120 of International Patent Application No. PCT/EP2024/056816, filed Mar. 14, 2024, which claims the benefit of German Patent Application No. 10 2023 106 504.1, filed Mar. 15, 2023, the contents of each of which are incorporated by reference herein.

The present invention relates to a medical microscope, in particular with an opto-mechanical system for flexibly adjusting a field of view of the microscope. The invention also relates to a microscope system.

In medical applications, microscopes are used for the examination and/or treatment of patients. They are referred to as medical microscopes, surgical (operation OP) microscopes, diagnostic microscopes, examination microscopes, or simply microscopes. Medical microscopes are used in particular in microsurgery, for example, in neurosurgery (surgery in the area of the head and intervertebral discs), ophthalmology (cataract surgery), plastic surgery (cosmetic surgery) or dental medicine (root canal treatment, implantology, . . . ). Medical microscopes are typically used for stereoscopically captured views of tissue to be examined or treated and provide significant, often adjustable, magnification factors.

A medical microscope comprises a binocular observation (e.g., Keplerian tube) in conjunction with, for example, a multi-stage switchable magnification changer according to Galileo (telescope system) in combination with coaxial illumination (e.g., with a 6 V/30 W incandescent lamp), which is fed into the microscope beam path coaxially via a mirror, for example. This basic design corresponds to the first surgical microscopes from 1953 by Dr. Littmann (Zeiss AG) and Prof. Wullstein (University of Würzburg), with which the first microsurgical operations in the ENT field were successfully performed. This was followed by the use of microscopes in ophthalmology, neurosurgery, gynecology, urology, and, at the end of the 1990s, in dental medicine.

Using a detachable coupling system, medical microscopes are held in place by a flexible support system, such as a ceiling, wall, or floor stand, or by a fixed support system.

The coupling system usually comprises a hanging mount mechanics that detachably connects the microscope to the support system and optionally can provide one or more degrees of freedom. Parts of the coupling system (herein also referred to as microscope hanging mount) are formed on the microscope and on the support system. One objective of the support system and the microscope hanging mount is to enable free positioning of the microscope in relation to the patient being examined and to provide the most rigid (fixed) positioning of the microscope possible during the examination/treatment so that a capturing of the field of view with the microscope can be performed that is as free from shake as possible.

The state of the art includes a wide variety of configurations of microscopes and microscope hanging mounts.

Optical configurations of microscopes relate, in particular, to the configuration of a microscope body and an ocular system. The microscope body comprises an objective system comprising, for example, a magnification system (e.g., a magnification changer such as a Galilean changer) and a main objective with one (or more) patient-side objective or main lens of the microscope. Depending on the application, magnification systems can provide several, for example 3-6,magnification levels or a ZOOM system with continuous magnification adjustment. The image of a focal plane (herein also referred to as microscope plane) is produced from two observation directions, so that two partial beam paths, which fall onto the focal plane at an angle, are to be guided through the microscope body and the ocular system. In the ocular system, the image from the objective system is further magnified using a binocular. The main objective focuses the binocular partial beam paths, which are spatially offset from an optical axis of symmetry, to form a common focus in the focal plane of the microscope. The main objective can be configured, for example, as a vario-objective with adjustable focal lengths. The ocular system comprises one or more ocular lenses with the respective tubes for each of the two partial beam paths and is also referred to as the observation tube. Typical magnifications of microscopes with binocular systems range from 2.5× to 30× or more.

The microscope hanging mount is usually adapted to the various types of stands used in medical environments. Hanging mount mechanics are common that engage on one side of the microscope and, thus, extend aside the microscope and that provide a degree of freedom of movement in the form of a forward and backward pivot movement of the microscope for positioning the field of view on the patient.

If the objective system and ocular system of a microscope are rigidly connected to each other, the operator must follow the movement of the microscope with his head when it is moved. Manufacturers of such microscopes include, in addition to the applicant Jadent GmbH, inter alia Zeiss AG, Leica Microsystems GmbH, Karl Kaps GmbH & Co. KG, and Global Surgical Corporation. Furthermore, there are known configurations of microscopes in which, while the ocular is fixed in space, the objective system can be moved and, thereby, the field of view can be relocated. This means that, for example, an operator does not have to change his posture when he changes the field of view in one direction. For example, DE 603 05 413 T2 discloses an optical system for adjusting a field of view in one direction, and U.S. Pat. No. 8,922,884 B2 discloses a microscope with a flexible objective lens arrangement with two axes of movement that extend perpendicular to each other and perpendicular to the beam path.

The inventors have recognized disadvantages of known microscopes, such as the complexity of the optical systems and the limited adjustability of the location of the field of view when the ocular is fixed in position. Thus, it was generally recognized that there is a need for microscopes, particularly low-cost microscopes, that allow the field of view to be (re)positioned as easily as possible during treatment/surgery.

One aspect of this disclosure is therefore based on the objective of providing a compact and cost-effective setup of a microscope system that allows flexible adjustment and/or change of the field of view—preferably in three-dimensional space. According to a further objective of one aspect of this disclosure, the operator of a microscope should be able to vary the field of view within a certain range—without changing an ergonomic body posture once it has been taken. In summary, the usability of a medical microscope is to be improved in such a way that physically stressful body postures of the operator are avoided/reduced, and a field of view can be changed in all directions in the focal plane without changing the ergonomic observation through the oculars. Such aspects can lead to increased ergonomics, quality, and safety during examinations/treatments of patients.

At least one of these objectives is solved by a medical microscope according to claim 1 and by a microscope system according to claim 10. Further developments are given in the dependent claims.

In one aspect, a medical microscope comprises a microscope ocular system with a binocular, an ocular base body holding the binocular, and a microscope hanging mount, which is applied to the ocular base body and is configured for attaching the medical microscope to a support system. Furthermore, the medical microscope comprises a microscope body with at least one objective lens for capturing light from a field of view, wherein the microscope body is assigned a microscope axis that corresponds to a section of an optical axis of symmetry extending from the microscope body to the field of view. The medical microscope further comprises an opto-mechanical system that is arranged between the microscope ocular system and the microscope body and that feeds light captured with the microscope body to the binocular, wherein the opto-mechanical system is configured for a pivot movement of the microscope body about a pivot axis and comprises a first rotation unit for a rotational movement of the microscope body about a rotation axis given by the first rotation unit, wherein the first rotation axis extends coaxially, parallel or at an angle in the range from 0° to 5° to a section of the optical axis of symmetry that extends through the first rotation unit.

In a further aspect, a microscope system comprises a support system, which is configured, in particular, as a floor, wall or ceiling stand or as a permanently mounted support system, and a medical microscope according to any one of the preceding claims, which is mounted to the support system with a microscope hanging mount of a microscope ocular system of the medical microscope.

In a further aspect, a medical microscope comprises a microscope ocular system with a binocular, an ocular base body holding the binocular, and a microscope hanging mount, which is applied to the ocular base body and is configured to attach the medical microscope to a support system. Furthermore, the medical microscope comprises a microscope body with at least one objective lens for capturing light from a field of view, wherein the microscope body is assigned a microscope axis that characterizes an observation direction extending from the microscope body to the field of view, as well as an opto-mechanical system for shifting the field of view in a microscope plane. The opto-mechanical system decouples movement of the microscope body from the microscope ocular system and the microscope hanging mount.

In some embodiments, the first axis of rotation can be oriented relative to the microscope axis in a basic setting of the microscope within an angle range from 25° to 100° or 45° to 85°, and/or the first axis of rotation, during a rotational movement around the first axis of rotation, an orientation of the first axis of rotation with respect to the microscope axis can remain unchanged. Furthermore, the pivot axis can be oriented relative to the microscope axis within an angular range from 75° to 105°, in particular orthogonally. On the microscope body, there can be provided, for example, hand grips for orienting the microscope body and shifting the field of view.

In some embodiments, the opto-mechanical system can comprise at least one pivot unit configured for the pivot movement of the microscope body around the pivot axis. Optionally, the pivot unit can be mounted to the ocular base body by means of the first rotation unit, so that the first rotation unit is configured for a rotational movement of the pivot unit about the first rotation axis and, in particular, during a pivot movement about the pivot axis, the orientation of the first rotation axis relative to the microscope axis changes, or the pivot unit can be mounted to the microscope body via an angle element, whereby, in particular, the first rotation unit can be arranged between the pivot unit and the angle element, so that, in particular, during a pivot movement around the pivot axis, the orientation of the first rotation axis to the microscope axis remains unchanged.

In some embodiments, for shifting the field of view, the opto-mechanical system can cause a decoupling of a movement of the microscope body from the microscope ocular system and, in particular, the microscope hanging mount.

In some embodiments, the opto-mechanical system can be configured such that the binocular forms a fixed point in three-dimensional space for an operator during setting of a location of the field of view by moving the microscope body, in particular about the pivot axis and/or about the first axis of rotation and/or about the second axis of rotation.

In some embodiments, using the pivot unit, the microscope axis can be settable within an angle range from 0° to ±20° relative to the course of the microscope axis in a basic setting of the microscope, and/or, using the first rotation unit, the microscope axis can be settable within an angle range from 0° to ±20° relative to a course of the microscope axis in a basic setting of the microscope.

In some embodiments, the pivot unit can be configured as device for deflecting the partial beam paths based on mirrors and/or prisms, which are movable relative to each other, and/or wherein the first rotation units can be configured as optical turntables.

In some embodiments, the microscope ocular system can be configured as a straight tube with a fixed observation direction in an angle range in the mounted state from 0° to 40° relative to a horizontal plane, or the microscope ocular system can further comprise an ocular pivot tube and/or an ocular rotation unit between the binocular and the ocular base body for adjusting an observation direction into the binocular.

In some embodiments, at least one magnetic fixation device for blocking movement about a corresponding axis can be provided for the pivot unit (and/or the first rotation unit), and/or the microscope hanging mount can be part of a ball joint system.

In some embodiments of the microscope system, the support system can engage the microscope hanging mount of the microscope ocular system, in particular, via a ball joint system, at an angle in the range from 0° to 20° with respect to a vertical direction, in particular, vertically from above. In further embodiments, the support system can engage the microscope hanging mount of the microscope ocular system at an angle in the range from 0° to 90° with respect to a vertical direction, in particular, obliquely from above.

In some embodiments of the microscope system, the microscope hanging mount of the microscope ocular system can be formed as part of a ball joint system.

In particular, it is one aspect of this disclosure to enable a change of the field of view by orienting the microscope unit in two directions while maintaining the position of the oculars in space (and, thus, the operator).

Spatially unrestricted positioning of the microscope, for example with electric drives on several axes, allows the operator to use both hands freely for the medical examination and/or treatment of a patient.

Disclosed herein are concepts that allow at least partial improvement of aspects from the prior art. In particular, further features and their usefulness will result from the following description of embodiments based on the figures. From the figures show:

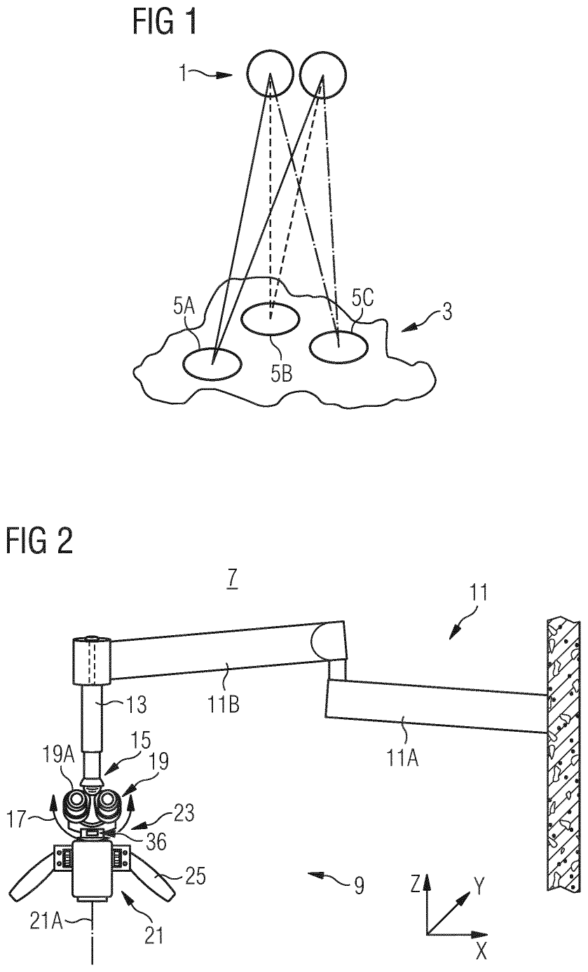

FIG. 1 a schematic representation for illustrating the inventive concept,

FIG. 2 a schematic representation of a microscope system,

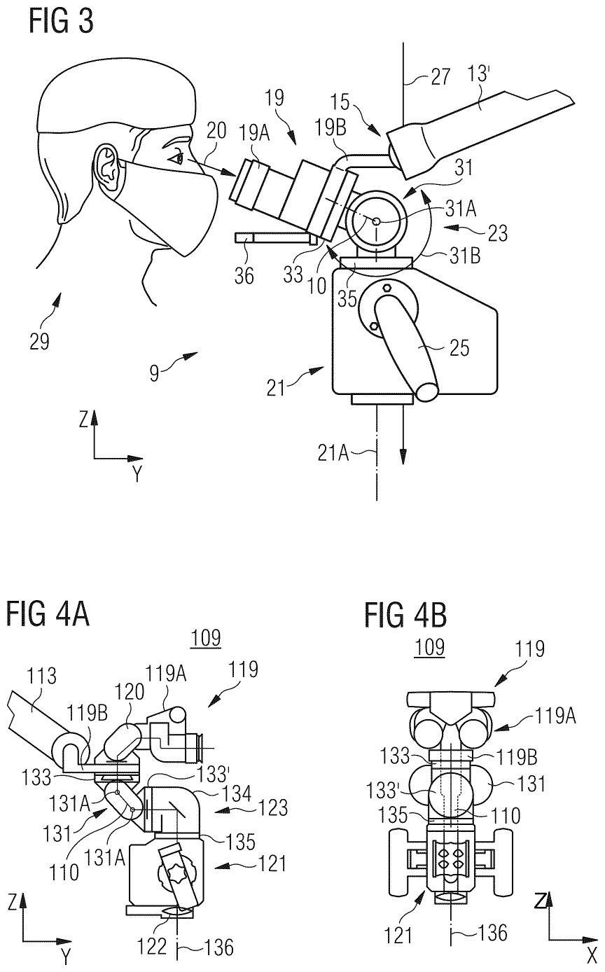

FIG. 3 a schematic representation on the use of a microscope according to the invention,

FIGS. 4A and 4B schematic representations of an extended implementation of the inventive concept in a medical microscope,

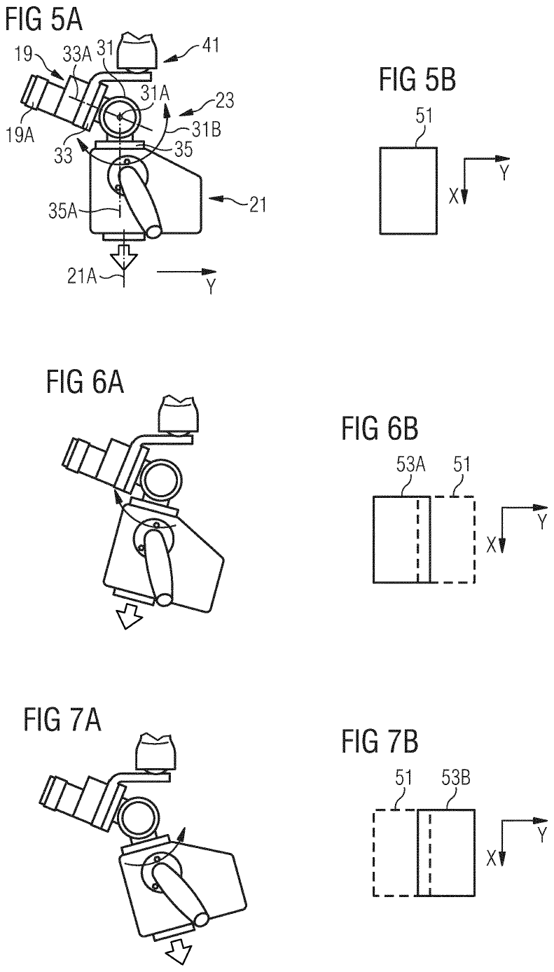

FIGS. 5A to 7B schematic representations for illustrating a shift of a field of view of a medical microscope by means of a pivot unit,

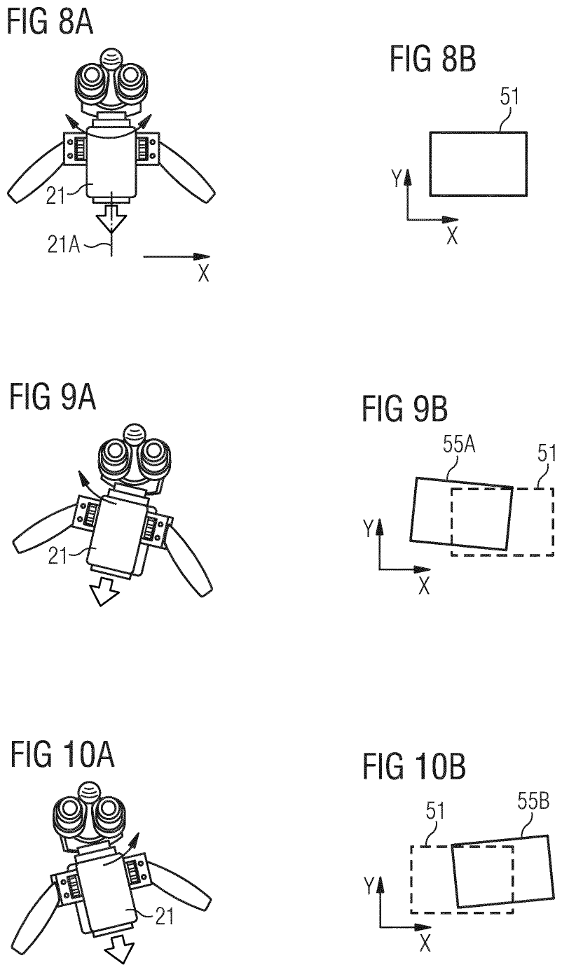

FIGS. 8A to 10B schematic representations for illustrating a shift of the field of view of a medical microscope by means of a first rotation unit, and

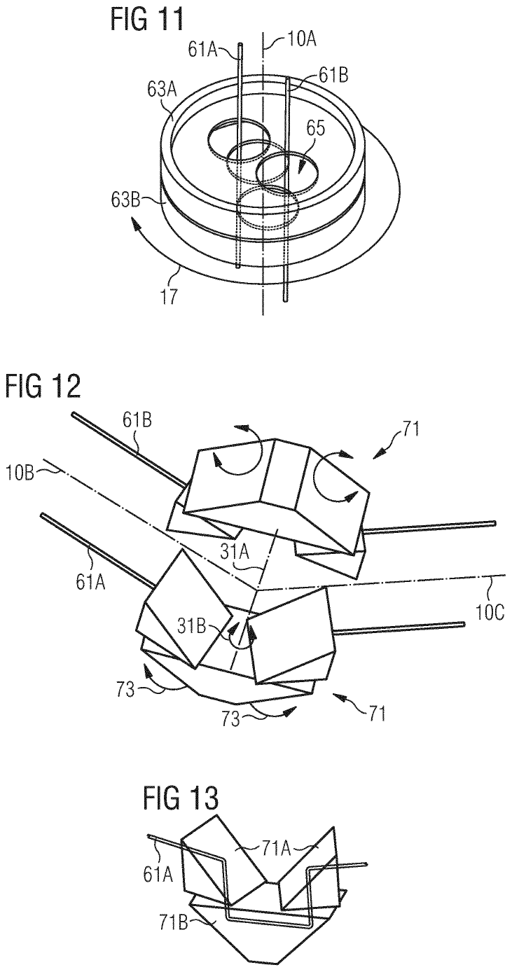

FIGS. 11 to 13 schematic representations for illustrating an exemplary rotation unit and an exemplary pivot unit.

The concepts proposed by the inventors enable shifts of the field of view in the focal plane/microscope plane, for example, by movements of the microscope body relative to two or more axes, in particular, pivot/tilt or rotation axes. For this purpose, an opto-mechanical decoupling of the partial beam paths between the ocular system and the objective system is performed.

As illustrated in FIG. 1, the decoupling is configured in such a way that an operator of the microscope can adjust to a fixed ocular position in space (fixed image point of the microscope) and can maintain this position even during a (fine motoric) movement of the objective system for relocating the field of view. FIG. 1 shows that, for the spatially fixed position of an ocular system (illustrated by two circles 1 representing a binocular), a field of view on a tissue 3 to be examined can be at several positions 5A, 5B, 5C. As a result, a change in the operator's posture is not required despite the change in the field of view.

FIG. 2 shows a microscope system 7 with a medical microscope 9 held by a support system 11. The support system 11 comprises, for example, a (ceiling, wall, floor) stand with a rear arm 11A and a spring arm 11B. The stand is configured such that the microscope 9 can be positioned as freely as possible (roughly) in space in height (Z-direction) as well as in the horizontal plane (X- and Y-direction) relative to a patient to be examined/treated (rough motoric movements). A direction of observation toward the patient to be examined/treated extends from the microscope 9, specifically the objective lens, to the field of view. The direction of observation is determined by a microscope axis 21A assigned to the microscope 9 (see also the optical axis of symmetry introduced below). Depending on the field of medicine, medical microscopes provide different working distances in the direction of observation. For example, the distance between a focal plane of the microscope extending perpendicular to the microscope axis 21A and a microscope body is in the range of f=175-200 mm in ophthalmology and in the range of f=300-420 mm in neurosurgery. In the example shown in FIG. 2, the microscope 9 is attached via a microscope hanging mount to a hanging mount device 13 that extends below the spring arm 11B in a vertical direction (or generally at an angle in the range from 0° (from above) to 20° with respect to a vertical direction). As an alternative to a fixed mount or a mount that can be moved around one or more defined axes, the microscope hanging mount shown in FIG. 2 comprises a ball joint system 15 that can provide a freely adjustable orientation of the mounted microscope 9, as illustrated by an arrow 17.

To illustrate another common mounting approach, FIG. 3 shows microscope 9 being held by a hanging mount device 13′that extends obliquely rearward and upward (for example, at an angle in the range from 0° to 90° to a vertical direction). The hanging mount device 13′can be implemented, for example, at an angle of 60° to the horizontal plane. Apart from this aspect of the hanging mount, the microscopes in FIGS. 2 and 3 are identical in terms of their ocular system and objective system.

The ball joint system 15 and the mechanics of the support system 11 can be locked (e.g., mechanically or magnetically). This allows further movement of the microscope 9 to be prevented once a desired position of the microscope 9 has been reached.

In FIGS. 2 and 3, there is also shown a binocular 19A of an ocular system 19 of the microscope 9. If the binocular 19A is firmly connected via a base body 19B and the microscope hanging mount (in this case, the ball joint system 15 with the support system 11 in the locked state), a fixed position in space of the binocular 19A is given. The microscope hanging mount, the base body 19B, and the binocular 19A form the ocular system 19 of the microscope 9. For a fixed positioning of the binocular 19A in space, the hereafter described inventive movability of a microscope body 21 of the microscope 9 relative to the ocular system 19 enables (fine) relocating of the field of view of the microscope 9 by orienting the microscope axis 21A. The invention is based on an opto-mechanical system 23 arranged between the microscope ocular system 19 and the microscope body 21. See, in particular, the side view of the microscope shown in FIG. 3.

In a basic setting assigned to the microscope 9, the orientation of the microscope axis 21A (and, thus, the focal plane) with respect to the binocular 19A (and, thus, with respect to the observation direction into the binocular 19A) and to the microscope hanging mount is set. For example, in the assembled case, the microscope axis 21A of the microscope 9 can extend vertically in the basic setting (i.e., the focal plane extends horizontally) and the observation direction can extend obliquely downward at an angle to the horizontal plane in the range from 10° to 45° or in the range from −10° to 50° or of ±10°.

If the binocular 19A and the microscope hanging mount are configured, for example, for a rigid connection to the support system 11, the orientation of the microscope axis 21A in space and relative to the binocular 19A is determined solely by the orientation of the microscope body 21 relative to the microscope hanging mount. The basic setting with a horizontal focal plane is then given, for example, by a specific setting of the microscope body 21 and the resulting orientation of the microscope axis 21A.

If the microscope hanging mount is provided with degrees of freedom of movement, the orientation of the microscope axis 21A in space derives additionally from the position of the microscope hanging mount. If the binocular 19A is also provided with degrees of freedom of movement, the orientation of the microscope axis 21A relative to the binocular 19A derives additionally from the orientation of the binocular 19A. A basic setting of the microscope will then usually be the orientation of the microscope body 21/the microscope axis 21A that is present in a preferred setting of the microscope hanging mount, whereby the preferred setting of the microscope hanging mount is given by a desired observation direction into the binocular 19A (potentially, for a predetermined orientation of the binocular 19A). For the embodiment shown in FIG. 2, in the basic setting, for example, the microscope 9 can be aligned with its direction of observation along (coaxial or parallel offset) the vertical hanging mount device 13. For the embodiment shown in FIG. 3 and described below, the basic setting can, for example, be selected such that, when the ball joint 15 is not locked, the direction of observation of the microscope 9 assumes a desired, for example vertical, orientation and the observation direction assumes a desired orientation to the horizontal plane, for example, at 20°.

Usually, the basic setting is in a middle range of a degree of freedom of movement provided by the opto-mechanical system 23 in order to provide the operator with a sufficient degree of movement in both directions of the degree of freedom starting from the basic setting.

To position the microscope body 21, one can grasp the microscope body 21, for example, at laterally provided hand grips 25.

Using the ball joint 15, orienting the microscope 9 by an operator 29, for example in a one-handed movement, about a vertically extending axis 27, which extends through a ball head of the ball joint system 15 and, e.g., the center of gravity. In combination with this, a fixed orientation of the observation direction into the binocular, for example at 20° or 45° with respect to the horizontal plane, can be provided. Alternatively, an adjustable angle relative to the horizontal plane can be provided for orienting the observation direction, see also the exemplary embodiment shown in FIGS. 4A and 4B having a binocular that is adjustable via an ocular pivot tube.

The hanging mounts shown in FIGS. 2 and 3 can be applied as central hanging mounts centrally to the microscope 9. In particular, a central hanging mount can be configured as a vertical hanging mount extending vertically upward, as shown in FIG. 2, which is applied in the middle (centrally) to the ocular system 19. Such hanging mounts make it possible that the view on the sides of the microscope body 9 is not restricted by protruding components of the hanging mount. Direct eye contact can contribute significantly to clearer communication and thus offers advantages in the course of the examination/treatment. A vertical hanging mount can also have the advantage that the mechanical axis of the vertical hanging mount and the microscope axis 21A (beam path after the main lens) can extend coaxially or parallel to each other in the basic setting of the microscope 9.

The opto-mechanical system 23 according to the invention decouples the location of the field of view of the microscope 9 from the position of the ocular system 19 in space. It is an aim to enable shifting the field of view in the focal plane by means of pivot and rotation (pivot) movements of the microscope body 21 while the ocular system 19 remains in a fixed position. The shifting of the field of view over the object to be examined/treated is carried out without the operator 29 having to change during the examination/treatment the posture that he assumed at the beginning of an examination/treatment with regard to the position of the ocular system 19 in space, which he has selected (see FIG. 1).

The shift of the field of view can be linearly, for example (illustrated exemplarily in FIG. 3 based on one degree of freedom of a pivot unit 31). For illustration, a pivot movement of the microscope body 21 around a pivot axis 31A is indicated by an arrow 31B in FIG. 3. The pivot movement is accompanied by a shifting of the field of view in the Y direction, whereby exemplarily in FIG. 3B the pivot axis 31A can be oriented orthogonally and generally within an angle range from 75° to 105° with respect to the microscope axis (21A).

Furthermore, the shift of the field of view can take place along a circular path (exemplarily in FIG. 3 based on a degree of freedom of a first rotation unit 33). The first rotation axis 33A can be oriented with respect to the microscope axis 21A in a basic setting of the microscope 9 in an angle range from 25° to 90°. It can also be seen that, during a rotational movement about the first rotation axis 33A, the orientation of the first rotation axis 33A relative to the microscope axis 21A remains unchanged. In FIG. 3, one can further see that the pivot unit 31 is mounted to the ocular base body 19B by means of the first rotation unit 33, so that the first rotation unit 33 is configured for a rotational movement of a pivot unit 31—and, thus, of the microscope body 21 mounted to the pivot unit 31—about the first rotation axis 33A. Accordingly, the orientation of the first rotation axis 33A relative to the microscope axis 21A changes during a pivot movement about the pivot axis 31A.

Exemplary shifts are explained below in connection with FIGS. 5A to 10B.

The opto-mechanical system 23 can introduce, as a further degree of freedom, a rotation of the microscope unit about an axis, for example, the optical axis of the microscope body 21, based on a degree of freedom of a second rotation unit 35 (see FIG. 4A). With this possibility of rotation of the microscope unit, the microscope unit can be rotated (e.g., for reasons of space or access, but in the embodiment of the rotation unit described herein without changing the captured image).

A combination of multidimensional shift and orientation of the field of view using the opto-mechanical system and the herein described hanging mount with a ball joint system (free adjustment according to degrees of freedom around three axes) can lead to a degree of flexibility, ergonomics, and ease of use that is not known in the prior art.

FIG. 3 also shows a mouth switch 36 that can be operated by the operator 29, for example, to control drives of the opto-mechanical system 23 and/or the locking of the ball joint system 15 or the support system 11 and/or an autofocus function. The use of an autofocus function is particularly advantageous with regard to the shift and orientation of the field of view using the opto-mechanical system because this enables rapid adjustment of the focal plane if it is no longer aligned with the tissue to be examined due to the shift and orientation of the field of view.

The opto-mechanical system 23 represents an interface that extends the microscope 9 in the area between the ocular system 19 and the microscope body 21 by one or more pivot (tilt) and/or rotation units. Specifically, the opto-mechanical system 23 should enable a flexible shift of the field of view in the focal plane of the microscope without the operator 29 having to change the preferred posture taken by the operator.

A pivot unit allows a pivot movement around a pivot axis (also referred to as tilting), whereby the pivot axis is transverse to a section of an optical axis of symmetry assigned to the pivot unit, the optical axis of symmetry being assigned to the partial beam paths. A rotation unit enables a rotational movement about a rotation axis, which is coaxial or parallel offset or essentially parallel, i.e., with an angular deviation of a few degrees, for example deviations in the range from 0° to 5°, to a section of the optical axis of symmetry associated with the rotation unit, the optical axis of symmetry being associated with the partial beam paths. For the opto-mechanical system, FIGS. 3, 4A, and 4B show exemplary sections of an optical axis of symmetry 10, 110, which in the embodiments extend centrally and orthogonally through the rotation units 33 and 35 and at an angle through the pivot units (see FIGS. 1 to 13).

Here, the optical axis of symmetry generally refers to the binocular partial beam paths that are guided through the microscope by a plurality of optical elements. The optical axis of symmetry characterizes the essential course of the optical beam guidance. Assuming a symmetrical course of the binocular partial beam paths, the optical axis of symmetry extends centrally between the partial beams. Sections of the optical axis of symmetry can be assigned to optical units; they extend from the entry into an optical unit to the exit.

Exemplary embodiments are given in connection with FIGS. 11 to 13.

While maintaining the ocular system 19 as a fixed point, the opto-mechanical system 23 can provide a shift range in the focal plane, i.e., in the figures in the X direction and in the Y direction, in which each deflection of the microscope axis from a “zero setting” according to the basic setting of the microscope (e.g., the vertical orientation in FIG. 2) comprises, for example, up to ±15° or more, in particular up to, for example, ±20°. In addition to a pivot movement with the pivot unit, a rotation unit can provide a rotation around the optical axis of symmetry of ±15° or more, in particular, up to, for example, ±20°. Depending on the orientation of the axis of rotation with respect to the microscope axis, the rotation unit can provide a shift on a curved path (rotation axis does not extend orthogonally to the microscope axis, see first rotation axis in FIG. 3, or extends parallel to the microscope axis with an offset) or a linear shift (rotation axis extends orthogonally to the microscope axis).

Within the range of deflection angles provided by the opto-mechanical system 23, the opto-mechanical system 23 preferably provides beam guidance, which is influenced as little as possible. Binocular turntables (as an example of a rotation unit) and binocular ocular pivot tubes (as an example of a pivot unit) are common optical components used in connection with the orientation of oculars. Essential to an aspect of the invention is the new use for decoupling the microscope optics in the case of a fixed ocular system, i.e., the movability of the microscope body relative to the microscope ocular system held stationary by the support system.

Rotational and/or pivot movements in the opto-mechanical system or of the support system can be mechanically guided. Thereby, axes of movement can be defined via gear wheels with as little play as possible and coordinated with each other for a change of direction. Furthermore, spring accumulators can be installed to balance the various axes of movement, whereby movements should preferably be ensured with balanced spring tension in all movement ranges. Despite a high degree of mobility around the axes, independent drifting away from a set position should be reduced/avoided as far as possible. In other words, a positioning of the microscope body set by the operator during an examination or treatment should remain stable for the required time. This can be additionally ensured by magnetic fixations and/or mechanical brakes on the axes. Alternatively or additionally, stepper motors can be provided for the axes of movement provided by the opto-mechanical system.

To reduce/suppress stray light in the opto-mechanical system 23, the beam-guiding arrangements of optical components, such as mirrors or prisms, can be supplemented with absorbing (e.g., anodized) components.

As mentioned above, the opto-mechanical system can enable the use of a straight tube in the ocular system, with the same comfort and significantly lower manufacturing costs, because an orientation/a height adjustment can be achieved via, for example, the ball joint of the vertical hanging mount disclosed herein.

The optical partial beam paths of a microscope extend through the microscope body with the main lens, the opto-mechanical system, and the binocular with the oculars and tubes. An optical axis of symmetry can be assigned section-wise to the partial beam paths, the optical axis of symmetry extending centrally between the partial beam paths. The optical axis of symmetry defines the overall course of the partial beam paths in a sequence of linear sections from the focal plane to the binocular and serves herein for the description of the orientations of pivot or rotation axes.

In FIGS. 4A and 4B, optical partial beam paths 108 are illustrated exemplarily for a further embodiment of a medical microscope 109 according to the invention with a more optically complex setup. For the opto-mechanical system, there is exemplarily indicated in FIGS. 4A and 4B an axis of symmetry 110 with respect to the two optical partial beam paths 108.

The microscope 109 comprises an adjustable ocular system 119, in which a binocular 119A is mounted to a microscope hanging mount 119B via an ocular pivot tube 120, so that there is flexibility in setting the observation direction into the binocular 119A. The microscope hanging mount 119B comprises a section, exemplarily formed in a plate shape, to which the ocular pivot tube 120 is attached on the top side. The microscope hanging mount 119B can be mounted to a boom 113 of the support system in such a way that the plate-shaped formed section remains essentially in a horizontal orientation when the microscope 109 is positioned above the patient.

An opto-mechanical system 123 is mounted onto the bottom side of the plate-shaped section, the opto-mechanical system 123 providing multiple axes of movement and degrees of freedom. The opto-mechanical system 123 comprises a pivot unit 131 with two pivot axes 131A. One or two rotation units 133, 133′are provided at the input and output of the opto-mechanical system 123, in particular, of the pivot unit 131. Furthermore, the opto-mechanical system 123 comprises a 90° deflection optic 134 (as an example of an angular optic element), which connects the rotation unit 133 to a microscope body 121 (with a main lens 122) via a further rotation unit 135. If the first rotation unit 133′is arranged between the pivot unit 131 and the angular element 134, the orientation of the first rotation axis with respect to the microscope axis 136 remains unchanged during a pivot movement about the pivot axis(axes) 131A.

The pivot unit 131 of the opto-mechanical system allows a shift of the field of view in the Y direction (pivot axes perpendicular to the associated section of the optical axis of symmetry). In the respective arrangement shown, the rotation units 133, 133′allow a shift of the field of view essentially in the X direction (rotation axes parallel offset or perpendicular to the microscope axis 121A) and can be used alternatively or together. Finally, the (optional) rotation unit 135 allows a rotation of the microscope unit.

To simplify the optical design, given the intended pivot movement of the microscope body 121, e.g., in the Y direction, a lower pivotability of the binocular 119A can be sufficient (see above notes on the possible use of a straight tube, e.g., with fixed observation directions in an angle range from 25° to 70° relative to a horizontal plane in the mounted stage of the microscope). In general, a provided orientability of the binocular 119A can be limited to an angle range, for example, to ranges from 25° to 70° or from +10° to +50° or from −10° to +50° or from −10° to +10°. Thus, the introduction of the opto-mechanical system 123, in particular, through the use of a straight binocular system in combination with the flexible hanging mount (e.g., based on a ball joint) disclosed herein, can allow for a more cost-effective realization of an ocular system 119.

FIGS. 5A, 6A, and 7A illustrate the pivotability of the microscope body 21 about the pivot axis 31A that is provided by the pivot unit 31 of the opto-mechanical system 23. Furthermore, FIG. 5A illustrates rotation axes 33A, 35A of the rotation units 33, 35 as well as a microscope axis 21A of the microscope body 21. The latter serve, in particular, for the illustration of the possibilities for shifting the field of view explained in connection with FIGS. 8A to 13B. Furthermore, FIG. 5A shows a hanging mount device 41 that is arranged vertically above the microscope, in particular, in the basic setting vertically above the center of gravity of the microscope.

FIG. 5A illustrates a basic setting of the microscope in which it has settled with its center of gravity below the hanging mount device 41. The angle settings of the opto-mechanical system 23 are selected in the basic setting, for example, such that the microscope axis 121A is directed vertically downward. This resulted in a horizontal focal plane of the microscope in the X-Y plane. The rotation axis 33A extends at an angle of approximately 70° (corresponding to the observation direction in the binocular 19A) with respect to the microscope axis 21A, and the rotation axis 35A extends with an offset parallel to the microscope axis 121A. The pivot axis 31A extends perpendicular to the associated section of the optical axis of symmetry (in the illustration in X direction).

In FIG. 5B, a schematic field of view 51 of the microscope body 21 is sketched exemplarily as a rectangle in the X-Y plane (usual fields of view are circular or oval) in order to illustrate also rotations of the field of view in the X-Y plane in addition to linear shifts of the field of view in the X-Y plane.

In FIGS. 6A and 7A, the microscope body 21 is pivoted toward the operator (toward the side of the binocular 19A) and away from the operator (away from the side of the binocular 19A), respectively, thereby shifting the field of view in the ±Y direction. This results in correspondingly linearly shifted fields of view 53A, 53B.

FIGS. 8A, 9A, and 10A illustrate a rotation of the microscope body 21 about the rotation axis 33A provided by the rotation unit 33 of the opto-mechanical system 23. (The rotation about the rotation axis 33A, which extends obliquely downwards into the drawing plane, is illustrated by arrows.)

FIG. 8A again shows the basic setting of the microscope and FIG. 8B shows the field of view 51 in the X-Y plane.

In FIGS. 9A and 10A, the microscope body 21 is shown rotated to the left and right, respectively. As the axis of rotation 33A extends not orthogonal with respect to the microscope axis 21A, on sees in FIGS. 9A and 10A a tilt of the microscope body 21. This leads to a shift of the field of view on a circular arc and, thus, to a rotation of the field of view. This results in correspondingly shifted and realigned fields of view 55A, 55B, see FIGS. 9B and 10B.

FIGS. 11 to 13 illustrate examples of implementations of the optical units as they can be used with an opto-mechanical unit 23 according to the invention. With regard to the structural components, the courses of the partial beam paths and the corresponding sections of the axis of symmetry are indicated.

FIG. 11 schematically shows a rotation unit that can be used as an interface, the rotation unit comprising two halves 63A, 63B that can rotate relative to each other about a section 10A of the axis of symmetry (arrow 17, see also FIG. 2). Each of the halves comprises a pair of openings 65 for the binocular partial beam paths 61A, 61B. The openings 65 are indicated exemplarily as circular in FIG. 11, but their shape is not limited and they can also be formed oval, angular, or banana-shaped extending around the axis of rotation (with additional apertures in the beam path, if necessary). The openings 65 restrict the acquired light that is assigned to the two partial beam paths 61A, 61B and transmitted by the rotation unit. In a basic setting of the microscope unit, the openings 65 can be aligned with each other, for example, in such a way that there is maximum overlap and, thus, minimum light loss, for example. In the rotation angle shown in FIG. 11, one can see that the pairs of openings 65 are rotated relative to each other, but the overlap of the openings is still large enough to allow sufficient image information to pass through. Depending on the required shift of the field of view (which also depends, inter alia, on the setup and working distance of the microscope), rotation angles in the range of ±20° (or more) or ±10° or ±5° in each direction of rotation can be provided with a tolerable loss of light (within the limits of a field of view restriction that does not limit the operation).

FIG. 12 schematically shows an exemplary optical setup of a pivot unit that can be used as an interface. One can see optical components 71A, 71B (e.g., mirrored prisms/roof prisms) of two deflection prism systems 71 assigned to partial beam paths 61A, 61B (see also FIG. 13 with respect to the course of the beam path 61A in the deflection prism system of the partial beam path 61A). The optical components 71 are held in a housing (not shown) that has a mechanism allowing the indicated rotations (arrows 73). The mechanism allows a deflection of the overall path of the partial beam paths 61A, 61B about a pivot axis 31A (see also FIG. 3). The deflection is accompanied by a pivoting of the microscope unit relative to the ocular optical system about the pivot axis 31A and is shown in FIG. 12 by an arrow 31B and an angled course of the axis of symmetry (sections 10B, 10C), whereby the pivot axis 31A extends orthogonally to the sections 10B, 10C of the axis of symmetry, for example. For example, the two rotations indicated for each of the partial beam paths can be coupled, in particular, performed in opposite directions. The optical components are configured (in as compact a structure as possible) and arranged relative to each other in such a way that, in the desired deflection angle range, the acquired light beam is guided essentially completely through the deflection prism systems 71. Deflection angles in the range of ±20° (or even more, up to several tens of degrees, can be possible), whereby also for the pivot unit, the deflection angle range and the shift of the field of view achievable with it also depend, inter alia, on the setup and working distance of the microscope,. Furthermore, the pivot unit can also have a sequence of such deflection prism systems for greater flexibility in beam guidance.

The use of prism systems (or, in general, mirror-based or prism and mirror combining setups) in the opto-mechanical system, in particular, in the context of the pivot unit, can also allow a reduction or even elimination of imaging errors (such as vignetting or cropping of the light beam) and of double images.

As a result, the opto-mechanical system allows free selection of the location of the field of view around the (basic) field of view 51 in the basic setting.

The preceding description illustrates the wide range of possibilities for shifting and orienting the field of view of the microscope body using the opto-mechanical systems disclosed herein. When using the microscope according to the invention, the result is a high level of ergonomics for the operator of the microscope. The concepts disclosed herein allow the operator, e.g., to assume an ergonomic sitting/standing position that can be maintained in a relaxed manner even during operations lasting several hours. The inventive concept allows that the binocular can remain spatially in the same place while still a larger field of view to be observed is enabled by pivoting and/or rotating the microscope unit. This is possible, in particular, because it is worked under high magnification, and, therefore, a small change in the orientation of the microscope unit (a small change in the angle of the microscope axis) leads to a sufficiently large image shift in the object field/focal plane.

It is explicitly emphasized that all features disclosed in the description and/or claims are to be considered separate and independent of each other for the purpose of the original disclosure as well as for the purpose of limiting the claimed invention, regardless of the combinations of features in the embodiments and/or claims. It is explicitly stated that all range indications or indications of groups of units disclose any possible intermediate value or subgroup of units for the purpose of the original disclosure as well as for the purpose of limiting the claimed invention, in particular, also as the limit of a range indication.

1. A medical microscope comprising:
a microscope ocular system with a binocular an ocular base body holding the binocular, and a microscope hanging mount, which is applied to the ocular base body and is configured to attach the medical microscope to a support system,
a microscope body with at least one objective lens for capturing light from a field of view, wherein the microscope body is associated with a microscope axis that corresponds to a section of an optical axis of symmetry extending from the microscope body to the field of view, and
an opto-mechanical system arranged between the microscope ocular system and the microscope body and feeding light captured with the microscope body to the binocular,
wherein the opto-mechanical system is configured for a pivot movement of the microscope body about a pivot axis and comprises a first rotation unit for a rotational movement of the microscope body about a first rotation axis given by the first rotation unit, wherein the first rotation axis extends coaxially, parallel or at an angle in the range from 0° to 5° to a section of the optical axis of symmetry that extends through the first rotation unit.

2. The medical microscope according to claim 1, wherein the first axis of rotation is oriented relative to the microscope axis in a basic setting of the microscope within an angular range from 25° to 100° or from 45° to 85°, and/or wherein, during a rotational movement about the first axis of rotation, an orientation of the first axis of rotation relative to the microscope axis remains unchanged, and/or wherein the pivot axis is oriented relative to the microscope axis within an angular range from 75° and 105°, in particular orthogonally.

3. The medical microscope according to claim 1, wherein the opto-mechanical system comprises at least one pivot unit, which is configured for the pivot movement of the microscope body about the pivot axis, and
wherein, optionally
the pivot unit is attached to the ocular base body by means of the first rotation unit, so that the first rotation unit is configured for a rotational movement of the pivot unit about the first rotation axis and that, in particular, in the case of a pivot movement about the pivot axis, the orientation of the first rotation axis changes relative to the microscope axis, or
the pivot unit is mounted to the microscope body via an angle element and the first rotation unit is arranged between the pivot unit and the angle element and, in particular, during a pivot movement about the pivot axis, the orientation of the first rotation axis relative to the microscope axis remains unchanged.

4. The medical microscope according to claim 1, wherein, for shifting the field of view, the opto-mechanical system causes a decoupling of a movement of the microscope body from the microscope ocular system and, in particular, from the microscope hanging mount.

5. The medical microscope according to claim 1, wherein the opto-mechanical system is configured such that the binocular forms a fixed point in three-dimensional space for an operator during setting of the location of the field of view by moving the microscope body, in particular, about the pivot axis and/or about the first axis of rotation and/or about the second axis of rotation.

6. The medical microscope according to claim 1, wherein, using the pivot unit, the microscope axis can be settable within an angle range from 0° to ±20° relative to a course of the microscope axis in a basic setting of the microscope, and/or
wherein, using the first rotation unit, the microscope axis can be settable within an angle range from 0° to ±20° relative to a course of the microscope axis in a basic setting of the microscope.

7. The medical microscope according to claim 1, wherein the pivot unit is configured as device for deflecting the partial beam paths based on mirrors and/or prisms, which are movable relative to each other and/or wherein the first rotation units is configured as optical turntable.

8. The medical microscope according to claim 1, wherein the microscope ocular system is configured as a straight tube with a fixed observation direction in an angle range in the mounted state from 0° to 40° relative to a horizontal plane, or
wherein the microscope ocular system further comprises an ocular pivot tube and/or an ocular rotation unit between the binocular and the ocular base body for setting an observation direction into the binocular.

9. The medical microscope according to claim 1, wherein at least one magnetic fixation device is provided for the pivot unit and/or the first rotation unit for blocking movement about a corresponding axis, and/or wherein the microscope hanging mount is configured as part of a ball joint system.

10. A microscope system comprising:
a support system, which is configured in particular as a floor, wall or ceiling stand or as a permanently mounted support system, and
a medical microscope according to claim 1, which is mounted to the support system with a microscope hanging mount of a microscope ocular system of the medical microscope

11. The microscope system according to claim 10, wherein the support system engages the microscope hanging mount of the microscope ocular system at an angle in the range from 0° to 20° to a vertical direction, in particular, vertically from above, in particular, via a ball joint system.

12. The microscope system according to claim 10, wherein the support system engages the microscope hanging mount of the microscope ocular system at an angle in the range from 0° to 90° with respect to a vertical direction, in particular, obliquely from above.

13. The microscope system according to claim 10, wherein the microscope hanging mount of the microscope ocular system is configured as part of a ball joint system.
